(19) United States
(12) Patent Application Publication
(10) Pub. No.: US 2008/0220444 A1
(43) Pub. Date: Sep. 11, 2008

(54) Bone Marrow Proliferation Assay

(76) Inventors: Michael S. Koratich, Springville, AL (US); Richard D. May, Vestavia Hills, AL (US)

Correspondence Address: Ballard Spahr Andrews & Ingersoll, LLP, SUITE 1000, 999 PEACHTREE STREET, ATLANTA, GA, 30309-3915, US

(21) Appl. No.: 10/570,973

(22) PCT Filed: Sep. 10, 2004
(86) PCT No.: PCT/US04/29628

§ 371 Date: Apr. 25, 2008

Related U.S. Application Data
(60) Provisional application No. 60/501,875, filed on Sep. 10, 2003.

(51) Int. Cl. G01N 33/53

(52) U.S. Cl. 435/7.2

(57) ABSTRACT

Disclosed herein are assay methods for assessing the myelotoxicity of a pharmacologic agent or a putative pharmacologic agent. Also, disclosed are kits and mixtures of cytokines and growth factors useful in the assay methods disclosed herein.

### CROSS-REFERENCE TO RELATED APPLICATIONS

This application claims benefit of priority from U.S. Provisional Application No. 60/501,875 filed Sep. 10, 2003, which is herein incorporated by reference in its entirety.

### STATEMENT REGARDING FEDERALLY SPONSORED RESEARCH

The disclosed subject matter was made with government support under Grant PO1 CA 34200 awarded by the National Cancer Institute. The US government has certain rights in the invention.

### FIELD

The disclosed subject matter relates generally to an assay for assessing the myelotoxic effect of pharmacologic agents or putative pharmacologic agents and to reagents used in the assay.

### BACKGROUND

Most anticancer and many anti-HIV drugs produce severe myelotoxicity, which may be dose limiting or may preclude entirely the use of a desire therapeutic agent. Prior to using a new drug clinically, it is important to evaluate its potential toxicity to hematopoietic cells of the bone marrow to establish starting dose levels for clinical trials. Myelotoxicity studies are often performed in vitro using labor-intensive colony-forming assays (e.g., CFU-GM). Specifically, testing has traditionally been designed to measure the effects of compounds on bone marrow (BM) progenitor cells in semi-solid medium for their growth to form colony-forming units/granulocyte-macrophage. The CFU-GM assay takes approximately 14 days from start to finish and is costly and labor-intensive, as colonies are counted manually by one technician. Needed in the art is a rapid, affordable myelotoxicity assay.

### SUMMARY

In accordance with the purpose(s) of the disclosed subject matter, as embodied and broadly described herein, disclosed herein, in one aspect, is an in vitro method of assaying myelotoxicity of an agent comprising the steps of activating bone marrow cells with a mixture of cytokines and growth factors; contacting the activated bone marrow cells with the agent to be tested; and detecting proliferation of the bone marrow cells in the presence of the agent. The disclosed subject matter further relates to kits and to mixtures of cytokines and growth factors useful in the described assay methods.

Additional advantages of the disclosed subject matter will be set forth in part in the description which follows, and in part will be obvious from the description, or may be learned by practice of the invention. The advantages of the disclosed subject matter will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects described below.

FIG. 1 is a graph of cell growth (percent of control where vehicle controls=100%) as a function of AZT concentration (μM). In FIG. 1, as in FIGS. 2-7, solid squares are data obtained from the proliferation assay disclosed herein and open squares are data obtained from the CFU-GM assay.

FIG. 2 is a graph of cell growth (percent of control where vehicle controls=100%) as a function of SN-38 concentration (μM).

FIG. 3 is a graph of cell growth percent of control where vehicle controls=100%) as a function of gemcitabine concentration (μM).

FIG. 4 is a graph of cell growth percent of control where vehicle controls=100%) as a function of thio-ara-C concentration (μM).

FIG. 5 is a graph of cell growth (percent of control where vehicle controls=100%) as a function of paclitaxel concentration (μM).

FIG. 6 is a graph of cell growth (percent of control where vehicle controls=100%) as a function of doxorubicin concentration (μM).

FIG. 7 is a graph of cell growth (percent of control where vehicle controls=100%) as a function of 4-HC concentration (μM).

### DETAILED DESCRIPTION

The disclosed materials, compositions, and methods may be understood more readily by reference to the following detailed description of specific aspects of the materials and methods and the Examples included therein and the previous and following description. Before the present materials, compositions, methods, articles, and/or devices are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a growth factor” includes mixtures of one or more growth factors, reference to “the cytokine” includes mixtures of two or more such cytokines, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “higher,” “enhances,” or “elevation” refer to increases above control levels, e.g., as compared to a basal level or as compared to an untreated control level. The terms “lower,” “inhibits,” or “reduction” refer to decreases below control levels, e.g., as compared to an untreated control level. By “control” is meant a cell sample in the absence of a particular variable such as a therapeutic or putative pharmacologic agent. Comparison to a control can include a comparison to a known control level or value known in the art.

The disclosed subject matter, in one aspect, provides a myelotoxicity assay. The purpose of this assay is to expedite the testing of potential myelotoxic drugs using bone marrow cells. The assay can be used to test the effect of any compound for its effects on human bone marrow, whether suppressive or enhancing. The assay expedites the process in that it takes only about 3 days from start to finish and relies on more automated means to measure the results (e.g., proliferation, measured by tritiated-thymidine uptake with a scintillation counter), thus reducing the labor and costs of the assay. Proliferation can be measured by other non-radioactive means, as well (e.g., vital dye conversion measured by spectrophotometry, 5-bromo-2-deoxyuridine (brdU) uptake measured by fluorescence, ATP quantitation by luminescence, etc.). The current disclosure utilizes tritiated-thymidine uptake to measure cell proliferation.

In another aspect, the disclosed subject matter relates to an in vitro method of assaying myelotoxicity of an agent comprising the steps of activating bone marrow cells with a mixture of cytokines and growth factors; contacting the activated bone marrow cells with the agent to be tested; and detecting proliferation of the bone marrow cells in the presence of the agent. A reduction in the amount of proliferation in the presence of the agent as compared to the amount of proliferation in the absence of the agent indicates the agent has a myelotoxic effect, whereas an increase in the amount of proliferation indicates that the agent is not myelotoxic, but rather enhances the growth of bone marrow cells.

The bone marrow cells used in the assay can be human. However, the bone marrow cells can be isolated from other species as well, including for example domesticated animals (e.g., cats, dogs, etc.), livestock (e.g., cattle, horses, pigs, sheep, goats, etc.), and laboratory animals (e.g., mouse, rabbit, rat, guinea pig, etc.). In the event bone marrow is used from other species, the cytokine mixtures would change from that described for the human example contained herein.

The assay method can further comprise enriching the bone marrow cells to a concentration of about 1×10⁵ to about 10×10⁵ per milliliter (ml) prior to the activation step. Methods of enriching bone marrow cells are known in the art.

In one aspect, the bone marrow cells are activated with a mixture of cytokines and growth factors. The mixture of cytokines and growth factors can optionally comprise at least two cytokines and growth factors selected from the group consisting of stem cell factor, granulocyte macrophage colony stimulating factor (GM-CSF), interleukin-3 (IL-3), interleukin (IL-6), and granulocyte colony stimulating factor (G-CSF). In another aspect, the mixture can comprise stem cell factor, GM-CSF, IL-3, IL-6, and G-CSF. In yet another aspect, the concentration of the stem cell factor can be at least about 20, 30, 40, 50, 60, 70, 80, 90, 100 ng/ml, where any of the stated values can form an upper and/or lower endpoint when appropriate, or any amount in between. In still another aspect, the concentration of stem cell factor can be about 50 ng/ml. The concentrations of the GM-CSF, IL-3, IL-6, and G-CSF can be, in one aspect, at least about 1, 10, 20, 30, 40, 50 ng/ml, where any of the stated values can form an upper and/or lower endpoint when appropriate, or any amount in between. In a further aspect, the concentration of one or more of the GM-CSF, IL-3, IL-6, and G-CSF can be about 20 ng/ml. Other cytokines can be used if the bone marrow progenitor cells of interest are precursors of other lineages. The example used herein is for myeloid cells of the granulocyte/macrophage (GM) lineage.

The activated bone marrow cells can then be contacted with an agent to be tested. Methods of contacting cells are known in the art and include, for example, immersing or spraying the cells with the agent, touching the agent to the cells, infusing the agent into the cell medium, and the like.

Proliferation can be detected by, for example, measuring the uptake of tritiated thymidine, measuring the uptake of a vital dye, measuring the incorporation of brdU (5-bromo-2-deoxyuridine), measuring the level of ATP generation, or other means known in the art at this time or later determined to be useful in assessing proliferation. A reduction in the amount of proliferation in the presence of the agent as compared to the amount of proliferation in the absence of the agent can indicate myelotoxicity.

The disclosed subject matter, in another aspect, further relates to a mixture of cytokines and growth factors as described herein. In one aspect, the mixture can comprise from about 20 to about 100 ng/ml of stem cell factor, from about 1 to about 50 ng/ml of GM-CSF, from about 1 to about 50 ng/ml of IL-3, from about 1 to about 50 ng/ml of IL-6, and from about 1 to about 50 ng/ml of G-CSF. More specifically, the mixture can comprise about 50 ng/ml of stem cell factor and about 20 ng/ml each of GM-CSF, IL-3, IL-6, and G-CSF. The disclosed subject matter can further comprise the mixture of cytokines and bone marrow cells in an in vitro system.

The disclosed subject matter also relates to a kit comprising a vessel or vessels containing the mixture disclosed herein or the individual growth factors and cytokines disclosed herein, optionally with instructions for use of the mixture in an in vitro method of assaying myelotoxicity of an agent, and optionally with bone marrow cells. The kit can comprise a vessel or vessels containing from about 20 to about 100 ng/ml of stem cell factor, from about 1 to about 50 ng/ml of GM-CSF, from about 1 to about 50 ng/ml of IL-3, from about 1 to about 50 ng/ml of IL-6, and from about 1 to about 50 ng/ml of G-CSF or other concentrations as described herein.

### EXAMPLES

The following examples are put forth so as to provide those of ordinary skill in the art with a complete disclosure and description of how the compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the yield and sensitivity obtained from the described processes. Only reasonable and routine experimentation will be required to optimize such process conditions.

### Example 1

### Validation of a Rapid Proliferation Assay to Assess Myelotoxicity as an Alternative to the Colony-Forming Assay (CFU-GM) with Human Bone Marrow

The myelotoxicity to human bone marrow of AZT (3′-azido-3′-deoxythymidine; Zidovudine), doxorubicin (Adriamycin), paclitaxel (Taxol), SN-38 (the active metabolite of irinotecan), thio-ara-C (OSI 7836), gemcitabine (Gemzar), and 4-hydroperoxycyclophosphamide (the preactivated form of cyclophosphamide (Cytoxan)) were assayed using the methods disclosed herein and compared to the results obtained with the CFU-GM assay. The results suggest that this new method can replace preliminary screening of potential anticancer or anti-HIV compounds during preclinical development and thus streamline this aspect of the drug development process.

A. Reagents and Materials

ROSETTESEP™ Bone Marrow Progenitor Cell Pre-Enrichment Cocktail (catalog number 15027 or 15067) was obtained from StemCell Technologies (Vancouver, BC, Canada). Dulbecco's phosphate-buffered saline (DPBS), RPMI 1640, fetal bovine serum (FBS), and sodium ethylenediaminetetraacetic acid (EDTA) was obtained from commercial sources (specific vendor information is unimportant). Density separation medium for human mononuclear cells (density of 1.077 g/mL) can be obtained from multiple vendors (HISTOPAQUE®-1077, obtained from Sigma Chemical Co., St. Louis, Mo., was used herein). 2-Mercaptoethanol and HEPES buffer were obtained from Sigma Chemical (St. Louis, Mo.). L-Glutamine and Antibiotic-Antimycotic were obtained from Invitrogen (Carlsbad, Calif.).

B. Preparation of Cells

Bone marrow (BM) was obtained from human donors, collected in Dulbecco's phosphate buffered saline (DPBS) containing 200 units/mL of heparin. The BM cells were then processed as described below. ROSETTESEP™ cocktail was added to the BM cells at a ratio of 50 μL of cocktail per 1 mL of cells in a test tube and the tubes were mixed well by repeated inversion. This mixture was then incubated at room temperature for approximately 20 minutes. A mixture containing PBS/2% FBS/1 mM EDTA was prepared and the BM sample was mixed in a 1:2 ratio (e.g., 10 mL marrow+20 mL PBS/2% FBS/1 mM EDTA) and gently mixed by inversion. The resultant mixture was layered over (or underlayed with) HISTOPAQUE®-1077 (or the equivalent) and centrifuged at room temperature for 25 minutes at 300×g with the brake off. The enriched BM cells were recovered from the HISTOPAQUE:plasma interface, washed twice with PBS/2% FBS/1 mM EDTA by centrifugation at 4° C. for 10 minutes at 270×g, and resuspended in complete medium (RPMI 1640 supplemented with 10% FBS, 25 mM HEPES, 50 μM 2-mercaptoethanol, 1 mM L-glutamine, and 1× Antibiotic-Antimycotic). Cells were counted and viability checked and cells were adjusted to 5×10⁵/mL in complete medium containing the following human recombinant growth factors/cytokines at the indicated final concentrations (cytokines may be obtained from various sources; those from StemCell Technologies were used herein):
Stem Cell Factor (50 ng/mL)
GM-CSF (20 ng/mL)
IL-3 (20 ng/mL)
IL-6 (20 ng/mL)
G-CSF (20 ng/mL)

C. Assay Procedure

Flat-bottom microtiter tissue culture plates (96 well) were used for the assay. Cells were placed in wells so that each well contained 5×10⁴ cells. Typically, cultures were performed in triplicate wells. Wells were brought to a final volume of 200 μL with either complete medium or various concentrations of test compounds, which were diluted in complete medium. Plates were covered with plastic wrap (to reduce evaporation) and placed in a humidified 5% CO₂ incubator at 37° C. for 3 days. Proliferation was measured by the uptake of tritiated-thymidine (³H-TdR) by pulsing wells with 1 μCi of ³H-TdR for the final 6-18 hours of incubation, harvesting the plate on a cell harvester (Brandel 96-well harvester) and measuring radioactivity on the filter mats in a scintillation counter (Wallac 1450 Microbeta TriLux). The effect of the test compounds were determined by comparing the results of cells treated with test compounds with control cells (cells stimulated with growth factors/cytokines and medium only).

D. Traditional CFU-GM Assay

Bone marrow was enriched after ficoll hypaque density centrifugation. 1×10⁵ bone marrow cells per dish, containing METHOCULT GF H4534 (1% methylcellulose, 30% FBS, 1% BSA, 10-4 M 2-ME, 2 mM L-gln, 50 ng/mL recombinant human (rh) stem cell factor (SCF), 10 ng/mL rh granulocyte/macrophage-colony stimulating factor (GM-CSF), and 10 ng/mL rh interleukin-3 (IL-3). METHOCULT was obtained from StemCell Technologies (Vancouver, BC, Canada). Cultures were treated with various concentrations of test compounds for the entire culture period. Cultures were performed in duplicate and were incubated for ˜14 days at 37° C. in 5% CO₂ and scored for colony growth (colony-forming units-granulocyte/macrophage, or CFU-GM) with a phase-contrast inverted microscope. Colonies containing >50 cells were considered positive.

E. Results

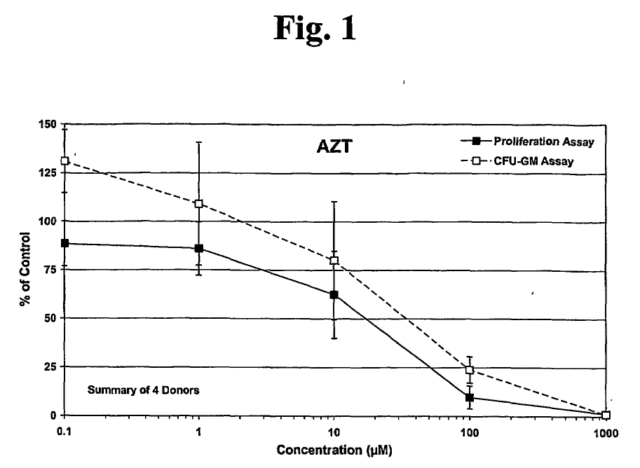

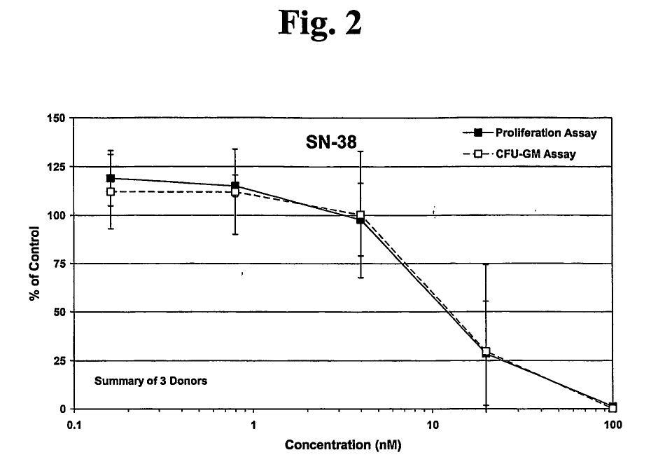

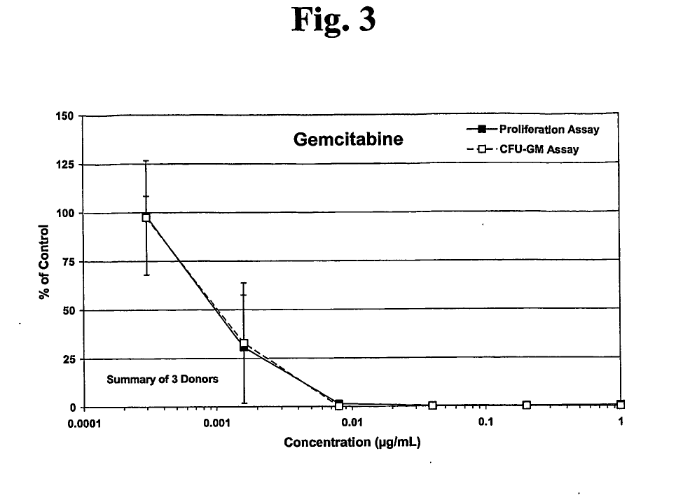

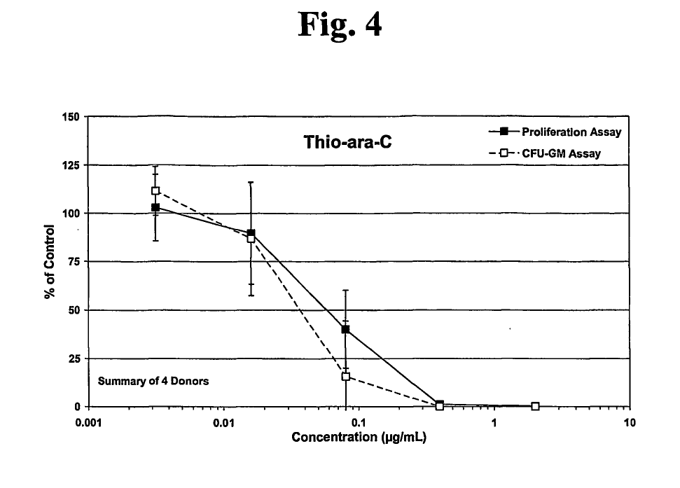

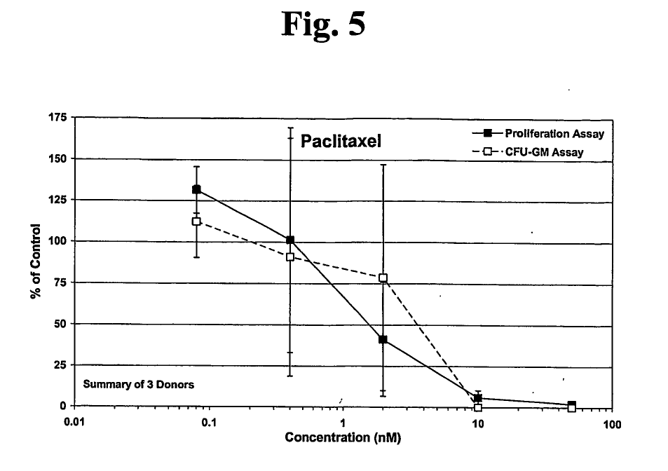

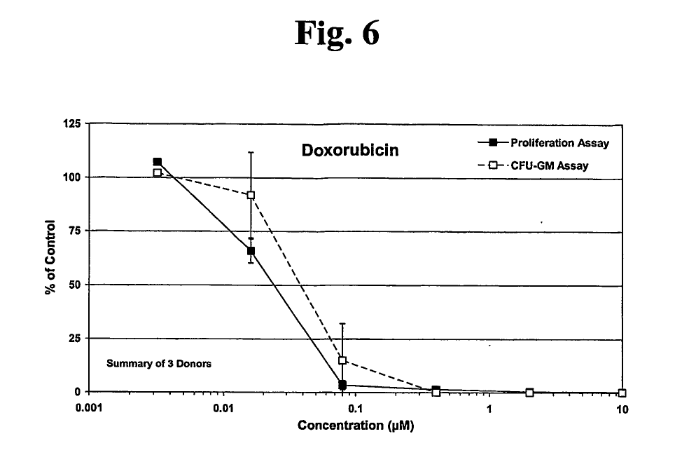

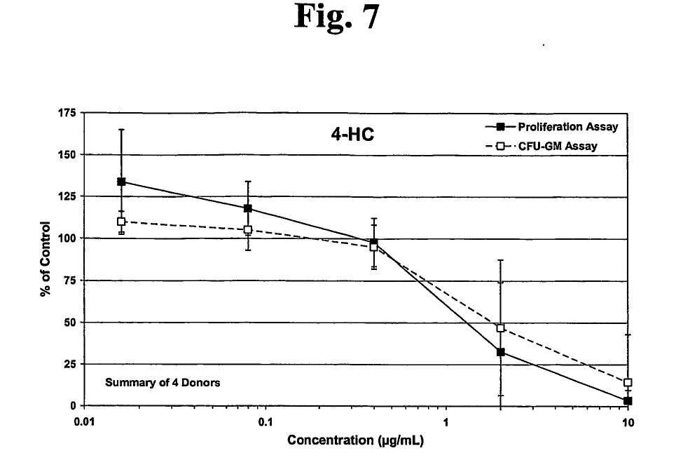

The proliferation assay disclosed herein took 3 days (as opposed to 14 days for the CFU-GM) to perform and cost much less (50-70%) than the CFU-GM assay. Data from the proliferation assay disclosed herein and the traditional CFU-GM assay were normalized to percent of control (vehicle controls=100%). These data are illustrated in FIGS. 1-7 for the various test compounds. The solid squares represent data obtained from the proliferation assay disclosed herein and the open squares represent data obtained from the traditional CFU-GM assay. The graphs show that there is no statistical difference between the proliferation assay disclosed herein and the traditional CFU-GM assay.

The concentrations that inhibited colony growth or proliferation by 50%, 75%, or 90% were determined by linear regression. These IC₅₀, IC₇₅, and IC₉₀ values for each of the various test compounds are provided in Table 1.

TABLE 1
IC₅₀, IC₇₅, and IC₉₀ values for various test compounds

| Drug | Assay | IC₅₀ | IC₇₅ | IC₉₀ |
|---|---|---|---|---|
| AZT | CFU-GM | 34.5 +/− 21.6 | 99.7 +/− 45.9 | 387.3 +/− 111.4 |
| | Prolif. | 17.1 +/− 12.4 | 50.4 +/− 17.3 | 145.8 +/− 66.2 |
| Gemcitabine | CFU-GM | 1.3 +/− 0.9 | 2.6 +/− 1.7 | 4.2 +/− 2.6 |
| | Prolif. | 1.2 +/− 0.6 | 2.6 +/− 1.5 | 4.4 +/− 2.6 |
| SN-38 | CFU-GM | 17.6 +/− 17.0 | 28.9 +/− 27.8 | 39.0 +/− 37.3 |
| | Prolif. | 14.5 +/− 9.3 | 28.7 +/− 20.5 | 48.7 +/− 30.1 |
| Thio-ara-C | CFU-GM | 40.3 +/− 42.3 | 79.5 +/− 82.3 | 122.0 +/− 121.4 |
| | Prolif. | 75.0 +/− 21.9 | 182.0 +/− 15.1 | 300.7 +/− 5.9 |
| Paclitaxel | CFU-GM | 3.4 +/− 2.8 | 4.9 +/− 3.9 | 6.0 +/− 4.7 |
| | Prolif. | 1.9 +/− 1.6 | 4.1 +/− 3.3 | 4.9 +/− 5.3 |
| Doxorubicin | CFU-GM | 40.0 +/− 14.0 | 77.3 +/− 41.8 | 131.7 +/− 103.4 |
| | Prolif. | 24.3 +/− 2.9 | 46.0 +/− 3.0 | 68.0 +/− 3.5 |
| 4-HC | CFU-GM | 1.5 +/− 0.9 | 3.1 +/− 1.9 | 5.1 +/− 3.1 |
| | Prolif. | 1.0 +/− 0.3 | 1.7 +/− 0.7 | 3.1 +/− 2.3 |

Comparing the toxicity of these 7 myelotoxic test compounds using the traditional CFU-GM assay and the proliferation assay disclosed herein reveals no statistical differences in the IC₅₀, IC₇₅, and IC₉₀ values between these two methods.

To evaluate the intra-assay variation, the IC₅₀, IC₇₅, and IC₉₀ values obtained when testing doxorubicin in the CFU-GM assay and the proliferation assay disclosed herein were compared. These values are provided in Table 2.

TABLE 2
Doxorubicin Intra-assay Variation

| Colony-forming Assay | | | Proliferation Assay | | |
|---|---|---|---|---|---|
| IC₅₀ | IC₇₅ | IC₉₀ | IC₅₀ | IC₇₅ | IC₉₀ |
| 25.7 | 47.4 | 68.3 | 12.3 | 32.6 | 64.7 |
| 31.0 | 50.8 | 68.2 | 23.1 | 47.1 | 72.1 |
| 22.4 | 44.6 | 67.6 | 25.5 | 48.7 | 71.7 |
| 32.2 | 52.1 | 69.5 | 25.9 | 48.7 | 71.2 |
| 34.2 | 54.6 | 72.4 | 26.2 | 49.0 | 71.2 |
| 29.1 | 49.9 | 69.2 | 22.6 | 45.2 | 70.2 |
| 4.9 | 3.9 | 1.9 | 5.9 | 7.1 | 3.1 |
| 16.8 | 7.9 | 2.8 | 26.0 | 15.7 | 4.4 |

The data show that the intra-assay variation, like the inter-assay variation, is similar between the two methods.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An in vitro method of assaying myelotoxicity of an agent comprising the steps of
(a) activating bone marrow cells with a mixture of cytokines and growth factors;
(b) contacting the activated bone marrow cells with the agent to be tested; and
(c) detecting proliferation of the bone marrow cells in the presence of the agent,
a reduction in the amount of proliferation in the presence of the agent as compared to the amount of proliferation in the absence of the agent indicating myelotoxicity.

2. The method of claim 1, wherein the bone marrow cells are human.

3. The method of claim 1, further comprising enriching the bone marrow cells to a concentration of 1×10⁵ to 10×10⁵ per ml prior to the activation step.

4. The method of claim 1, wherein the mixture of cytokines and growth factors comprises at least two cytokines and growth factors selected from the group consisting of stem cell factor, GM-CSF, IL-3, IL-6, and G-CSF.

5. The method of claim 4, wherein the mixture of cytokines and growth factors comprises stem cell factor, GM-CSF, IL-3, IL-6, and G-CSF.

6. The method of claim 4, wherein the concentration of the stem cell factor is 20-100 ng/ml.

7. The method of claim 4, wherein the concentration of the GM-CSF is from about 1 to about 50 ng/ml.

8. The method of claim 4, wherein the concentration of the IL-3 is from about 1 to about 50 ng/ml.

9. The method of claim 4, wherein the concentration of the IL-6 is from about 1 to about 50 ng/ml.

10. The method of claim 4, wherein the concentration of the G-CSF is from about 1 to about 50 ng/ml.

11. The method of claim 1, wherein the detection step comprises measuring the uptake of tritiated thymidine.

12. The method of claim 1, wherein the detection step comprises measuring the uptake of a vital dye.

13. The method of claim 1, wherein the detection step comprises measuring the incorporation of brdU.

14. The method of claim 1, wherein the detection step comprises measuring the level of ATP generation.

15. A mixture of cytokines and growth factors comprising from about 20 to about 100 ng/ml of stem cell factor, from about 1 to about 50 ng/ml of GM-CSF, from about 1 to about 50 ng/ml of IL-3, from about 1 to about 50 ng/ml of IL-6, and from about 1 to about 50 ng/ml of G-CSF.

16. A kit comprising a vessel containing the mixture of claim 15.

17. The kit of claim 16, further comprising instruction for use of the mixture in an in vitro method of assaying myelotoxicity of an agent.

18. A kit comprising a vessel or vessels containing from about 20 to about 100 ng/ml of stem cell factor, from about 1 to about 50 ng/ml of GM-CSF, from about 1 to about 50 ng/ml of IL-3, from about 1 to about 50 ng/ml of IL-6, and from about 1 to about 50 ng/ml of G-CSF.

19. The kit of claim 18, further comprising instruction for use of the stem cell factor, GM-CSF, IL-3, IL-6, and G-CSF in an in vitro method of assaying myelotoxicity of an agent.
